(19) United States
(12) Patent Application Publication
(10) Pub. No.: US 2026/0091221 A1
(43) Pub. Date: Apr. 2, 2026

(54) TRANDUCER ARRAY WITH CONFORMAL-SHAPED ELECTRICAL CONNECTIONS

(71) Applicant: Novocure GmbH, Baar (CH)

(72) Inventors: Noa HALAVEE, Tel Aviv (IL); Elie YAACOBI, Haifa (IL); Dmitry GOLOM, Haifa (IL)

(73) Assignee: Novocure GmbH, Baar (CH)

(21) Appl. No.: 19/344,197

(22) Filed: Sep. 29, 2025

Related U.S. Application Data
(60) Provisional application No. 63/701,262, filed on Sep. 30, 2024.

(51) Int. Cl. A61N 1/04

(52) U.S. Cl. CPC A61N 1/0476; A61N 1/0496

(57) ABSTRACT

A transducer apparatus for delivering tumor treating fields includes: an array of electrode elements, the array configured to be positioned over a subject's body with a front face facing the subject's body; electrical connections connecting the electrode elements and having a front face facing the subject's body and a back face; and an anisotropic material layer electrically coupled to the array of electrode elements and located on a front side of the front face of the array, the anisotropic material layer comprising a front face and a back face, the back face facing the array of electrode elements, wherein, when viewed in a direction perpendicular to and toward the back face of the anisotropic material layer, the electrode elements have convex shapes with respect to a perimeter of the anisotropic material layer and the electrical connections have concave shapes with respect to the perimeter of the anisotropic material layer.

### CROSS-REFERENCE TO RELATED APPLICATIONS

This application claims priority to U.S. Provisional Patent Application No. 63/701,262, filed Sep. 30, 2024, which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into a region of interest by electrode assemblies (also known as “transducer arrays” or “transducers”) placed on the patient's body and applying alternating current (AC) voltages between the transducers. Conventionally, transducers used to generate TTFields include a plurality of electrode elements comprising ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include rectangular arrays of ceramic disks aligned with each other in straight rows and columns and attached to the subject's body via adhesive.

### BRIEF DESCRIPTION OF THE DRAWINGS

FIGS. 1A and 1B depict a top plan view (back face) and a bottom view (front face), respectively, of an example transducer apparatus according to some embodiments.

FIG. 1C depicts a cross-section view of the example transducer apparatus of FIGS. 1A and 1B taken across section 1C-1C′ according to some embodiments.

FIG. 1D depicts a cross-section view of the example transducer apparatus of FIGS. 1A and 1B taken across section 1D-1D′ according to some embodiments.

FIG. 2 depicts a top plan view (back face) of example transducer apparatuses according to some embodiments.

FIG. 3 is a flowchart depicting an example of applying TTFields to a subject's body according to some embodiments.

Various embodiments are described in detail below with reference to the accompanying drawings, wherein like reference numerals represent like elements, and wherein descriptions of like elements may not be repeated for every embodiment, but may be considered to be the same if previously described herein.

The figures provided herein are for illustrative purposes and may not be to scale. Variations in dimensions, proportions, and configurations may exist between the figures and the actual embodiments. The figures are intended to facilitate understanding of the embodiments and should not be construed as limiting the scope of the disclosure.

### DESCRIPTION OF EMBODIMENTS

This application describes exemplary transducer apparatuses used to apply TTFields to a subject's body, for example, for treating one or more cancers. This application also describes exemplary methods to apply TTFields to a subject's body using transducers.

Transducers used to apply TTFields to a subject's body often include multiple electrode elements electrically coupled together on a substrate and attached to the subject's body at a desired location, for example, via an adhesive backing of the substrate or a separately applied adhesive. Conventional transducers have large, rectangular surfaces so as to maximize a number of electrode elements that are located on the transducer for applying TTFields to the subject's body. However, subjects can experience skin irritation on portions of their skin that are contacted by the electrode elements during TTFields treatment. Such irritation may be common at positions directly underneath the electrode elements, where heat and current may be at their highest concentrations, particularly for electrodes around the outer edge of the array. Additionally, a subject's body is typically non-planar over a large area and may bend and change shape as the subject moves. As such, the shape of conventional transducers can have difficulty adhering to a subject's body due to the contour of the placement area and bi-direction tension, resulting in a risk of detachment of the transducer apparatus when the subject shifts or moves.

As recognized by the inventors, on transducer arrays that comprise multiple electrode elements, the portions of the transducer arrays positioned directly beneath the electrode elements may become hotter than the portions of the transducer arrays positioned between the electrode elements. Furthermore, higher currents flow through the electrode elements that may be located along the edge of the array compared to the electrode elements located toward the middle of the array. Further still, an electrode element located at a corner or similar sharp bend in the edge of the array may have a higher current than other electrode elements along the edge and near the center of the array.

As recognized by the inventors, an uneven distribution of current through the transducer array may lead to higher temperature zones (or “hot spots”), e.g., at the corners or edges of the transducer array, which, in turn, may limit the maximum operational current that may be driven by a transducer array and, as a result, the strength of the resulting TTFields.

As recognized by the inventors, a transducer apparatus may also include an anisotropic material layer located on a side of the array of electrode elements facing the subject's body. Such an anisotropic material layer may spread the heat and/or current generated at the individual electrode elements within a plane that is perpendicular to the direction from the electrode elements to the subject's body. Spreading heat and/or current in this plane may reduce the concentration of heat and/or current at locations directly under the individual electrode elements, thus reducing the amount or severity of irritation, if any, that occurs on the subject's skin.

As further recognized by the inventors, a transducer apparatus including an anisotropic material layer may cause irritation to the subject due to the rigidness of the anisotropic material layer. For example, the anisotropic material layer may be relatively inflexible, like a sturdy piece of cardboard, and as such, may not easily conform to a subject's body, which is typically non-planar over a large area or which may bend and change shape as the subject moves. Moreover, due to a non-planar surface on a subject and/or a surface of the subject bending and changing shape, the anisotropic material layer may deform or even crack, resulting in the transducer array producing a less than desired electric field. The inventors have discovered that using a hole in, or substantially in, the center or the middle of the anisotropic material layer may help to alleviate this problem.

As further recognized by the inventors, a transducer apparatus including an anisotropic material layer may cause irritation to the subject due to the sharp edges of the anisotropic material layer. When a piece of anisotropic material is cut to size to be used as an anisotropic material layer in a transducer apparatus, the edges of the transducer apparatus may be sharp. As such, when these edges come in contact with a subject, the subject may experience discomfort. Due to a desire by the subject to reduce this discomfort from the transducer apparatus, the transducer apparatus may be placed in a different non-desired position on the subject for delivery of TTFields, which may result in the subject receiving less than a desired dosage of TTFields. Further, due to a desire by the subject to reduce this discomfort from the transducer apparatus, the subject may decrease the period(s) of time spent using the transducer apparatus, which may result in the subject receiving less than a desired dosage of TTFields. The inventors have discovered that using a flexible and compressible layer, for example a foam material, with the anisotropic material layer (for example, in locations on the skin-facing side of the anisotropic material layer) may help to alleviate this discomfort experienced by a subject.

The inventors have further discovered that the flexible and compressible layer may advantageously provide some additional flexibility for the array when the subject moves or twists the body part associated with the location of the array. Further, the added flexibility is enhanced when the flexible and compressible layer covers (on the skin-facing side) a central hole in the anisotropic material layer.

As further recognized by the inventors, the least flexible areas of the transducer apparatus are regions where the thickness of the anisotropic material layer is combined with the thickness of the electrode elements and a flexible printed circuit board (PCB). It was found that a transducer apparatus including curved electrode elements and a curved flexible PCB to electrically couple the curved electrode elements, where the curved electrode elements and/or the curved flexible PCB conform to the shape of the transducer array, may improve the flexibility of the transducer apparatus thereby allowing the apparatus to better conform to the contours of the subject's body and any twisting/bending of the subject's body. The curved flexible PCB may include: curved PCB pad(s) covering and electrically connected to the curved electrode elements; and curved electrical connection(s) (also referred to herein as PCB bridging connector(s)) electrically connected to the curved PCB pads. The curved electrode elements and curved flexible PCB may result in more comfort and/or less discomfort to the subject and/or may result in a fuller dose of TTFields being administered to the subject.

Descriptions of embodiments related to specific exemplary drawings herein may be applicable, and may be combined with, descriptions of embodiments related to other exemplary drawings herein unless otherwise indicated herein or otherwise clearly contradicted by context.

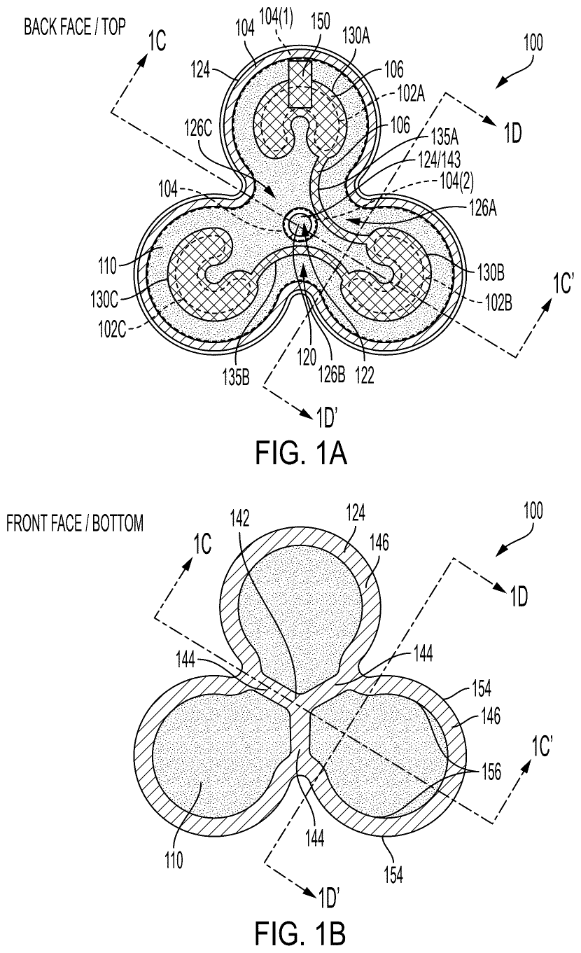

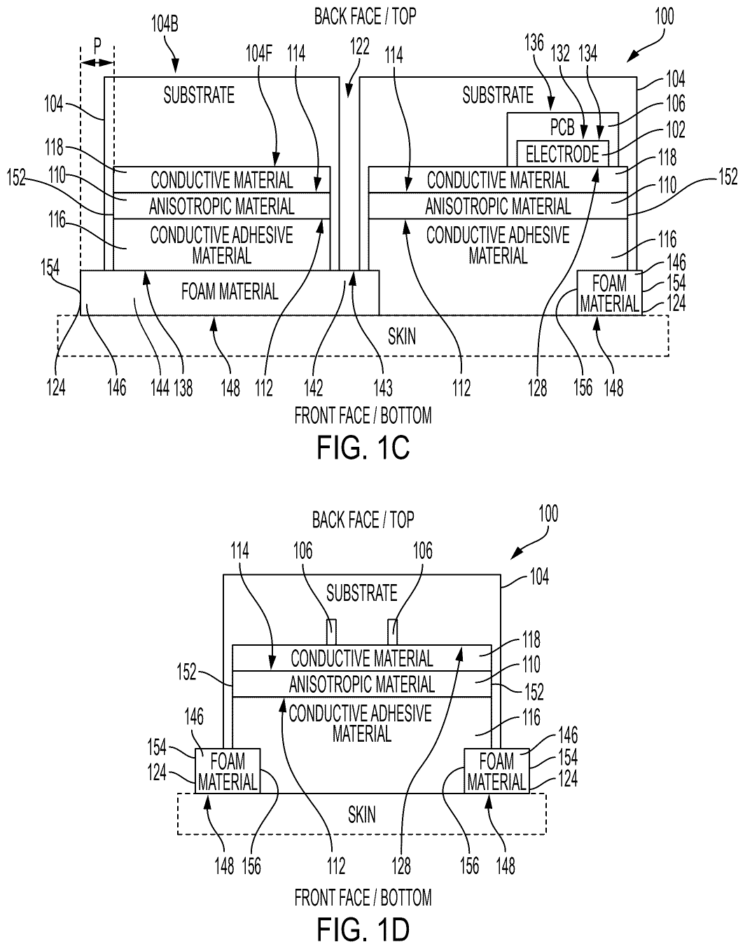

FIGS. 1A and 1B depict a top plan view (back face) and a bottom view (skin-facing, front face), respectively, of an example transducer apparatus 100 according to some embodiments. FIG. 1C depicts a cross-section view of the example transducer apparatus 100 of FIGS. 1A and 1B taken across section 1C-1C′ according to some embodiments, and FIG. 1D depicts a cross-section view of the example transducer apparatus 100 of FIGS. 1A and 1B taken across section 1D-1D′ according to some embodiments. FIGS. 1A and 1B illustrate the transducer apparatus 100 as viewed from a direction perpendicular to a face of the transducer apparatus 100. FIG. 1A illustrates a plan view from the back side of the transducer apparatus 100 (i.e., the side facing away from the subject's body, or the back face, or the top). FIG. 1B illustrates the front side of the transducer apparatus 100 (i.e., the side facing the subject's body, or the front face, or the bottom).

The transducer apparatus 100 may include at least two electrode elements 102 (i.e., 102A, 102B, 102C), a flexible printed circuit board (PCB) 106 electrically connecting the electrode elements 102, an anisotropic material layer 110 electrically coupled to the electrode elements 102, and a substrate 104 for holding the electrode elements and the anisotropic material layer against the subject's body. In FIG. 1A, the electrode elements 102A, 102B, and 102C are shown in dashed outline, as they are located between the flexible PCB 106 and the anisotropic material layer 110. In FIG. 1A, although the substrate 104 covers the back side of the transducer apparatus 100, the substrate 104 is shown as a cut-away in partial view by the dashed lines 104(1), 104(2) so as to illustrate the components beneath the substrate 104. As illustrated in FIGS. 1C and 1D, the substrate 104 covers the back side of transducer apparatus 100.

When viewed in a direction perpendicular to and toward the back face of the transducer apparatus 100, the transducer apparatus 100 may have a plurality of lobes (e.g., each having a circular shape or a substantially circular shape) extending from a center 120 or a shape comprised of a plurality of substantially circular shapes connected about the center 120. In some embodiments, each of the lobes may have a shape similar to the shape of a ping pong paddle (also known as a table tennis bat). In some embodiments, the center 120 of transducer apparatus 100 may be, for example, a geometric center of the transducer apparatus 100 or a centroid of the transducer apparatus 100. The shape may permit the transducer apparatus 100 to more readily conform to the subject's body, for example, as the subject twists and/or bends.

The transducer apparatus 100 may include at least two electrode elements 102. In some embodiments, the transducer apparatus 100 may have exactly three electrode elements 102. The plurality of electrodes elements 102 may be spaced about a center 120 of the transducer apparatus 100.

The electrodes elements 102 may be adapted to administer TTFields therapy to a subject. The electrodes elements 102 may be electrically conductive and may be substantially flat. The electrode elements 102 may or may not be capacitively coupled. The electrode elements 102 may be metal, metal alloy, layered metal, laminated conductive material, ceramic or non-ceramic dielectric material positioned over a flat conductor (e.g., polymer film disposed on flat metal). In some embodiments, the electrode elements do not have a dielectric material.

In some embodiments, the dielectric material of the electrode elements 102 may have a dielectric constant ranging from 10 to 50,000. In some embodiments, the layer of dielectric material may comprise a high dielectric polymer material such as poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CTFE)” and “Poly(VDF-TrFE-CFE),” respectively. The dielectric constant of these materials is on the order of 40. In some embodiments, the polymer layer may be poly(vinylidene fluoride-trifluoroethylene-chlorotrifluoroethylene-chlorofluoroethylene) or “Poly(VDF-TrFE-CTFE-CFE).” In some embodiments, the layer of dielectric material of the electrode elements 102 comprises a terpolymer comprising polymerized units of monomers such as VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer.

In some embodiments, when viewed in a direction perpendicular to and toward the back face of the subassemblies, the electrode elements 102 may conform to convex shapes of the perimeter of the anisotropic material layer 110. Each electrode element 102 may, or may not, have a same size and convex shape. The electrodes elements 102 may have a same shape or a substantially similar shape. In some embodiments, when viewed in a direction perpendicular to and toward the back face of the subassemblies, the electrodes elements 102 may be u-shaped, substantially u-shaped, c-shaped, substantially c-shaped, rounded v-shaped, substantially rounded v-shaped, jelly bean shaped, substantially jelly bean shaped, kidney bean shaped, substantially kidney bean shaped, horseshoe shaped, substantially horseshoe shaped, circular shaped, substantially circular shaped, oval shaped, substantially oval shaped, ovoid shaped, or substantially ovoid shaped.

In some embodiments, the end points of a given u-shaped (or similar shaped) electrode element 102 may be close together. In some embodiments, the end points of a given u-shaped (or similar shaped) electrode element 102 may have a gap in-between the end points. In some embodiments, an open end of a given u-shaped (or similar shaped) electrode element 102 may face the center 120 of the transducer apparatus 100, and an apex of the given u-shaped (or similar shaped) electrode element 102 may be farthest from the center 120.

In some embodiments, the electrode elements 102 may be located with respect to the center 120 of the transducer apparatus 100. For example, a centroid of each electrode element 102 may be spaced substantially equidistant (which includes equidistant) from the center 120 of the transducer apparatus 100. The electrode elements 102 may be spaced substantially equidistant (which includes equidistant) from each other about the center 120 of the array. The electrode elements 102 may be spaced substantially equidistant (which includes equidistant) from the center 120 of the array and/or may be spaced substantially equidistant (which includes equidistant) from each other.

The flexible PCB 106 may electrically couple together the electrode elements 102. The flexible PCB 106 may have a front face 134 facing the subject's body and a back face 136 opposite the front face 134 (FIG. 1C). In some embodiments, when viewed in a direction perpendicular to and toward the back face of the transducer apparatus 100, the flexible PCB 106 may cover the back faces 132 of the electrode elements 102. An edge of the electrode element 102 may be offset inwardly from an edge of the flexible PCB 106. In some embodiments, when viewed in a direction perpendicular to and toward the back face of the transducer apparatus 100, the flexible PCB 106 may cover up to 15%, 20%, 30%, 40%, 60%, 80%, or any percentage therebetween of a back face 114 of the anisotropic material layer 110. In some embodiments, flexible PCB 106 may not function as an electrode. In some embodiments, electrode elements 102 may be attached to or positioned on the flexible PCB 106. In other embodiments, the electrode elements 102 may be a component of or positioned within the flexible PCB 106. The flexible PCB 106 may be a non-adhesive region. In some embodiments, the flexible PCB 106 may comprise a polyimide film, such as, for example, Kapton® (from DuPont de Nemours, Inc., Wilmington, DE, USA) and conductive traces (for example, and without limitation, copper traces or traces of conductive ink, etc.).

In some embodiments, when viewed in a direction perpendicular to and toward the back face of the subassemblies, the flexible PCB 106 may have curved PCB pads 130 (i.e., 130A, 130B, 130C) and curved PCB bridging connectors (or electrical connections) 135 (i.e., 135A, 135B). A PCB pad 130 may be positioned on the back face 132 of an electrode element 102. A PCB pad 130 may be larger than the respective electrode element 102 facing the front face of the PCB pad 130. In some embodiments, each PCB pad 130 may have substantially the same shape as the electrode element 102 it is positioned over. The PCB bridging connectors 135 may be thinner than the PCB pads 130. The PCB pads 130 may be connected by the PCB bridging connectors 135. The PCB bridging connectors 135 may be electrical connections connecting the PCB pads 130 to each other. The PCB bridging connectors 135 may be electrical connections connecting the electrode elements 102 to each other. One PCB bridging connector 135 may electrically connect two PCB pads 130. For example, PCB bridging connector 135A electrically connects PCB pads 130A and 130B, and PCB bridging connector 135B electrically connects PCB pads 130B and 130C. One PCB bridging connector 135 may electrically connect two electrode elements 102. For example, PCB bridging connector 135A electrically connects electrode elements 102A and 102B, and PCB bridging connector 135B electrically connects electrode elements 102B and 102C. Three electrode elements 102 may be electrically connected by two PCB bridging connectors 135. For example, PCB bridging connectors 135A and 135B electrically connects electrode elements 102A, 102B, and 102C. In some embodiments, three electrode elements 102 may be electrically connected by three PCB bridging connectors 135.

In some embodiments, when viewed in a direction perpendicular to and toward the back face of the transducer apparatus 100, the PCB bridging connectors 135 may have a curved shape. The curved shape of the PCB bridging connectors 135 may permit the transducer apparatus 100 to more readily conform to the subject's body and to twisting movements of the subject's body. The PCB bridging connectors 135 may have concave shapes with respect to a perimeter of the anisotropic material layer 110. The PCB bridging connectors 135 may conform to concave shapes of the perimeter of the anisotropic material layer 110. The PCB bridging connectors 135 may have a shape mirroring the perimeter of the anisotropic material layer 110. The apexes of the concave shapes of the PCB bridging connectors 135 may be closer to the center of the anisotropic material layer 110 than the end points of the convex shapes of the electrode elements 102.

In some embodiments, when viewed in a direction perpendicular to and toward the back face of the transducer apparatus 100, the electrode elements 102 and/or the PCB pads 130 may conform to convex shapes of a perimeter of the anisotropic material layer 110, and the PCB bridging connectors 135 may conform to concave shapes of the perimeter of the anisotropic material layer 110. Each electrode element 102 may have a same convex shape and a same size, each PCB pad 130 may have a same convex shape and a same size, and each PCB bridging connector 135 may have a same concave shape and a same size. In some embodiments, when viewed in a direction perpendicular to and toward the back face of the transducer apparatus 100, the PCB pads 130 may have a same shape but larger size than the electrode elements 102.

In some embodiments, when viewed in a direction perpendicular to and toward the back face of the transducer apparatus 100, one of the PCB bridging connectors 135 having a concave shape may electrically connect two of the electrode elements 102 having convex shapes. In some embodiments, when viewed in a direction perpendicular to and toward the back face of the transducer apparatus 100, three electrode elements 102 having convex shapes may be electrically connected by two PCB bridging connectors 135 having concave shapes.

In some embodiments, when viewed in a direction perpendicular to and toward the back face of the transducer apparatus 100, the PCB pads 130 and the PCB bridging connectors 135 together may form an undulating shape. For example, the PCB pads 130 and the PCB bridging connectors 135 together may alternate between a convex shape and a concave shape.

The undulating shape of the PCB pads 130 and the PCB bridging connectors 135 may follow a perimeter of the transducer apparatus 100, a perimeter of the anisotropic material layer 110, and/or a perimeter of the substrate 104. The PCB bridging connectors 135 may include apexes of the undulating shape closest to a center of the anisotropic material layer 110, and the PCB pads 130 and/or the electrode elements 102 may include apexes of the undulating shape farthest from a center of the anisotropic material layer 110.

The flexible PCB 106 may be electrically connected to a PCB connector 150. When placed on a subject, the transducer apparatus 100 may be electrically coupled to a voltage generator (not shown) via the PCB connector 150.

In some embodiments, the transducer apparatus 100 may have one or more temperature sensors (such as, for example, thermistors) (not shown) associated with some or all electrode elements 102. Each of the one or more temperature sensors may be coupled to an electrode element 102. The temperature sensor may monitor the heat generated during treatment by measuring the temperature. The temperature sensor may help prevent overheating from occurring during treatment by alerting the subject or the caregiver when unsafe temperatures are being reached; thereby minimizing harm occurring to the subject. In some embodiments, temperature measurements may be collected at a controller which can instruct the AC generator to reduce current supplied to all electrode elements thereby reducing the temperature at the electrode elements. In some embodiments, temperature measurements may be collected at a controller which can instruct the AC generator to reduce the current supplied to specific one or more electrode elements to reduce the temperature at that specific one or more electrode elements.

Each of the one or more temperature sensors may be electrically connected via the flexible PCB 106. A portion of the flexible PCB 106 electrically connected to the temperature sensor may have a tentacle shape, rectangular shape, substantially rectangular shape, cylindrical shape, substantially cylindrical shape, circular shape, oval shape, ovoid shape, substantially circular shape, substantially oval shape, or substantially ovoid shape.

The transducer apparatus 100 may include one or more blank spaces 126 (or void space) (i.e., 126A, 126B, 126C), which do not overlap with any of the electrode elements 102. At least part of one or more of the blank spaces 126 may be a relief region for the subject since no electrode elements 102 are in the blank spaces 126. The blank spaces 126 may each be located between two adjacent electrode elements and around the center 120. In some embodiments, the transducer apparatus 100 may have an alternating pattern of electrode elements 102 and blank spaces 126.

The anisotropic material layer 110 of the transducer apparatus 100 may be directly or indirectly electrically coupled to the plurality of electrode elements 102 and may be located on a front face 128 of the electrode elements 102. In some embodiments, a conductive material 118, such as an outer layer of conductive adhesive material, may electrically couple the anisotropic material layer 110 of the transducer apparatus 100 to the plurality of electrode elements 102.

The anisotropic material layer 110 may be disposed over the plurality of electrode elements 102 such that, when viewed in a direction perpendicular to and toward the front face of the transducer apparatus 100, the anisotropic material layer 110 covers the electrode elements 102 and, optionally, the at least one blank space 126. In some embodiments, the anisotropic material layer 110 may be disposed over the plurality of electrodes to cover the electrode elements 102 and blank space 126. In some embodiments, when viewed in a direction perpendicular to and toward the back face of the transducer apparatus 100, the anisotropic material layer 110 may not extend outward all the way to the edge of the substrate 104.

The anisotropic material layer 110 may optionally include a hole 122 in, or substantially in, the center 120 or the middle of the anisotropic material layer 110, passing through a front face 112 and a back face 114, facilitating flexibility and pliability of the anisotropic material layer 110. The hole 122 may be circular, oval, or ovoid, or substantially circular oval, or ovoid in shape. In some embodiments, the hole 122 may be triangular, substantially triangular, rounded-triangular, or trilobal in shape. In some embodiments, the hole 122 may be rectangular, square, squoval, or an elongated slot in shape, The hole 122 may be positioned at or near the center 120 of the transducer apparatus 100. For example, the center 120 of the transducer apparatus 100 may be positioned at the center of the hole 122. In some embodiments, the hole 122 may be positioned offset from the center 120. Furthermore, the centroid of each electrode element 102 may be spaced substantially equidistant from the hole 122.

When viewed in a direction perpendicular to and toward the back face of the transducer apparatus 100, the flexible PCB 106 does not pass over the hole 122 of the anisotropic material layer 110 and/or may be positioned equidistantly from the hole 122 of the anisotropic material layer 110. The convex shapes of the electrode elements 102 may be away from the hole 122 of the anisotropic material layer 110. The convex shapes of the PCB pads 130 may be away from the hole 122 of the anisotropic material layer 110. The concave shapes of the PCB bridging connectors 135 may be towards the hole 122 of the anisotropic material layer 110. None of the electrode elements 102 and none of the flexible PCB 106 may cover the hole 122 of the anisotropic material layer 110.

In some embodiments, when viewed in a direction perpendicular to and toward the back face of the transducer apparatus 100, a perimeter of the anisotropic material layer 110 may have a repeating pattern of a convex side and a concave side about a center of the anisotropic material layer, which may be the same as the center 120 of the transducer apparatus 100. An apex of the concave side may be closer to the center 120 and/or the hole 122 than the apexes of the convex sides. For example, the perimeter of the anisotropic material layer 110 may include three convex sides and three concave sides, with the convex side and concave side alternating, and include three lobes (for example, as illustrated in FIGS. 1A and 1B).

In some embodiments, when viewed in a direction perpendicular to and toward the back face of the transducer apparatus 100, the anisotropic material layer 110 may have a plurality of lobes about the center of the anisotropic material layer. The perimeter of each lobe of the anisotropic material layer 110 may have a convex shape with respect to the perimeter of the anisotropic material layer 110. The lobes may be equidistant or substantially equidistant around the center of the anisotropic material layer. The lobes may be circular shaped, substantially circular shaped, oval shaped, substantially oval shaped, ovoid shaped, or substantially ovoid shaped. A perimeter of the anisotropic material layer 110 between the perimeter of each lobe of the anisotropic material layer 110 may have a concave shape with respect to the perimeter of the anisotropic material layer 110.

In some embodiments, when viewed in a direction perpendicular to and toward the back face of the transducer apparatus 100, each lobe of the anisotropic material layer 110 may include one of the electrode elements 102, and one of the PCB pads 130 (where included). The flexible PCB 106 may cover each electrode element 102 and extend between the lobes to electrically couple the electrode elements 102 positioned within the respective lobes. A PCB bridging connector 135 may be situated between two of the lobes.

In some embodiments, when viewed in a direction perpendicular to and toward the back face of the transducer apparatus 100, the electrode elements 102 and the flexible PCB 106 may be shaped to conform to the shape of the anisotropic material layer 110. For example, each electrode element 102 may have a convex shape conforming with a convex side of the anisotropic material layer 110, each PCB pad 130 of the flexible PCB 106 may have a convex shape conforming with a convex side of the anisotropic material layer 110, and each PCB bridging connector 135 of the flexible PCB 106 may have a concave shape conforming with a concave side of the anisotropic material layer 110.

The transducer apparatus 100 may further include a foam material layer 124 directly or indirectly coupled to the anisotropic material layer 110, located on the front face 112 of the anisotropic material layer 110, and configured to contact the subject's body. The foam material layer 124 may be positioned between a front face 138 of the conductive adhesive material 116 and the subject's body. A front face 148 of the foam material layer 124 may include an adhesive to aid in securing the transducer apparatus 100 to the subject's skin. In some embodiments, the adhesive is a biocompatible skin-contact adhesive. When viewed in a direction perpendicular to and toward the front face of the transducer apparatus 100, the foam material layer 124 may have an empty center such that the foam material layer 124 frames the anisotropic material layer 110, including the lobes of the anisotropic material layer 110. In some embodiments, the foam material layer 124 may be a continuous layer that covers some or most of the front face 112 of the anisotropic material layer 110 but does not cover all of the front face 112 of the anisotropic material layer 110. In some embodiments, the anisotropic material layer 110 may not extend outward all the way to the edge of the foam material layer 124. The foam material layer 124 may cover at most 10%, or at most 20%, or at most 30%, or at most 40%, or at most 50%, or any percentages therebetween, of the anisotropic material layer 110.

When viewed in a direction perpendicular to and toward the front face of the transducer apparatus 100, the foam material layer 124 may include a perimeter portion 146 covering a perimeter 152 (or outer perimeter, outer edge, or edge) of the anisotropic material layer 110. When viewed in a direction perpendicular to and toward the front face of the transducer apparatus 100, the perimeter portion 146 of the foam material layer 124 may include an outer perimeter 154 (or outer edge) and an inner edge 156 (or inner edge). In some embodiments, when viewed in a direction perpendicular to and toward the front face of the transducer apparatus 100, the perimeter portion 146 of the foam material layer 124 may cover up to the perimeter 152 of the anisotropic material layer 110. In some embodiments, the outer perimeter 154 of the perimeter portion 146 of the foam material layer 124 may be larger than the perimeter 152 of the anisotropic material layer 110. The perimeter portion 146 of the foam material layer 124 may have substantially the same shape as the perimeter 152 of the anisotropic material layer 110. In some embodiments, such as illustrated in FIG. 1C, an offset distance P between the perimeter 152 of the anisotropic material layer 110 and the outer perimeter 154 of the perimeter portion 146 of the foam material layer 124 may be the same about the perimeter of the transducer apparatus 100. Alternatively, in some embodiments, the offset distance P between the perimeter 152 of the anisotropic material layer 110 and the perimeter 154 of the perimeter portion 146 of the foam material layer 124 may vary about the perimeter of the transducer apparatus 100.

When viewed in a direction perpendicular to and toward the front face of the transducer apparatus 100, the foam material layer 124 may cover the hole 122 in the anisotropic material layer 110. In some embodiments, the foam material layer 124 may include a hole-covering portion 142, positioned over the hole 122 in the anisotropic material layer 110. The hole-covering portion 142 may be dimensioned larger than the hole 122 in the anisotropic material layer 110. A back face 143 of the hole-covering portion 142 of the foam material layer 124 may not be in contact with any other part or portion of the transducer apparatus 100. By being free of contact on the back face 143, the transducer apparatus 100 may be more pliable against a subject's body

When viewed in a direction perpendicular to and toward the front face of the transducer apparatus 100, the foam material layer 124 may include at least one connection portion 144 connecting the hole-covering portion 142 and the perimeter portion 146. The at least one connection portion 144 may cover the front face 112 of the anisotropic material layer 110. When viewed from a direction perpendicular to the front face 112 of the anisotropic material layer 110, the at least one connection portion 144 may be located coincident with the blank spaces 126, except on the skin-facing side of the anisotropic material layer 110.

The foam material layer 124 may facilitate a comfortable surface against the subject's body. In some embodiments, a transducer apparatus 100 having the anisotropic material layer 110 may include the foam material layer 124 to provide a conformable material in contact with the subject's body. The foam material layer 124 may be in the same plane as the conductive adhesive layer 116 so that the electrode elements 102 are placed as close as possible to the subject's skin, without the area of the electrode elements 102 of the transducer apparatus 100 projecting past the foam material layer 124.

In some embodiments, the foam material layer 124 may be at least 1 mm and at most 10 mm thick, or at least 2 mm and at most 10 mm thick, or at least 5 mm and at most 10 mm thick, or at least 8 mm and at most 10 mm thick. In some embodiments, the foam material layer 124 may be formed at least partially of a soft material with airy open space. In some embodiments, the foam material layer 124 may be formed of a non-porous material to improve sterilizability and cleanability. In some embodiments, the foam material layer 124 may comprise one or more of low-density polyethylene (LDPE), silicone, polyurethane, or ethylene-vinyl acetate (EVA), each of which may be an open-cell, closed-cell, or partially open/closed-cell foam.

In some embodiments, the foam material layer 124 may be an adhesive coated foam, which may include adhesive on one or both sides of the foam. In particular, the foam material layer 124 may include adhesive on the side facing the subject's body. In some embodiments, the adhesive on the skin-facing side facing the subject's body is a biocompatible skin-contact adhesive. In some embodiments, the foam material layer 124 may be a Tegaderm™ product, such as 3M™ Tegaderm™ Transparent Film Dressing Frame (3M, Saint Paul, MN, USA).

The substrate 104 may be configured for attaching the transducer apparatus 100 to a subject's body and may be configured to secure the various components of the transducer apparatus 100. The substrate 104 may hold the electrode elements 102 and the anisotropic material layer 110 against the subject's body. The substrate 104 may include a front face 104F facing the subject's body and a back face 104B opposite the front face 104F. The front face 104F of the substrate 104 may face the electrode elements 102 and the anisotropic material layer 110. The substrate 104 may have an outer perimeter extending beyond an outer edge of the anisotropic material layer 110. The substrate 104 may be contoured to match a shape of the outer edge of the respective anisotropic material layer 110. In some embodiments, the substrate 104 may not directly contact the subject's skin due to the foam material layer 124. Suitable materials for the substrate 104 may include, for example, tape, bandage, cloth, nonwoven fabric, foam, flexible plastic, and/or a conductive medical gel. The transducer apparatus 100 may be affixed to the subject's body via the substrate 104 (e.g., via an adhesive layer) and/or a conductive medical gel. The substrate 104 may be an adhesive bandage. In some embodiments, the adhesive may be a biocompatible adhesive. The substrate 104 may aid in securing a cable (not shown) connected to the flexible PCB 106 for providing signals to the flexible PCB 106 via the PCB connector 150 for generating TTFields with the transducer apparatus 100.

In some embodiments, when viewed in a direction perpendicular to and toward the back face of the transducer apparatus 100, the electrode elements 102 and the flexible PCB 106 may be shaped to conform to the shape of the substrate 104. For example, each electrode element 102 may have a convex shape conforming with a convex side of the substrate 104, each PCB pad 130 of the flexible PCB 106 may have a convex shape conforming with a convex side of the substrate 104, and each PCB bridging connector 135 of the flexible PCB 106 may have a concave shape conforming with a concave side of the substrate 104.

In some embodiments, the hole 122 may be through the substrate 104, the anisotropic material layer 110, the conductive adhesive material 116, and the conductive material 118, such that the hole 122 extends through each layer located in the same position but may not be the same size through each layer. The hole 122 may be in the center or substantially in the center of the transducer apparatus 100. In some embodiments, the hole 122 may be circular, oval, or ovoid shaped, or substantially circular, oval, or ovoid shaped. In some embodiments, the hole 122 may be at least 0.5 cm and at most 3.0 cm in diameter. For example, the hole 122 may be 0.5 cm, 1.0 cm, 1.5 cm, 2.0 cm, 2.5 cm, or 3.0 cm in diameter. In some embodiments, the hole 122 may be at least 0.5% to at most 5.0% of the area of the anisotropic material layer 110. For example, the hole 122 may be 0.5%, 1.0%, 1.5%, 2.0%, 2.5%, 3.0%, 3.5%, 4.0%, 4.5%, or 5.0% of the area of the anisotropic material layer 110. In some embodiments, the hole 122 may be at least 0.5% to at most 10%, or at most 20%, or at most 30%, or at most 40%, or at most 50%, of the area of the anisotropic material layer 110. In some embodiments, the hole 122 may be at least 0.5%, or at least 10%, or at least 20%, or at least 30%, or at least 40%, to at most 50%, of the area of the anisotropic material layer 110. In some embodiments, the hole in the substrate 104 may have a diameter smaller than the hole 122 of the anisotropic material layer 110. In some embodiments, the hole in the substrate 104 may have a diameter greater than the hole 122 of the anisotropic material layer 110. In some embodiments, the substrate 104 may not include a hole and covers the hole 122.

The anisotropic material layer 110 may have anisotropic thermal properties and/or anisotropic electrical properties. If the anisotropic material layer 110 has anisotropic thermal properties (for example, greater thermal conductivity in the plane of the layer than through the plane of the layer), then the layer spreads the heat out more evenly over a larger surface area. If the anisotropic material layer 110 has anisotropic electrical properties (for example, greater electrical conductivity in the plane of the layer than through the plane of the layer), then the layer spreads the current out more evenly over a larger surface area. In each case, this may lower the temperature of the hot spots and may raise the temperature of the cooler regions when a given AC voltage is applied to the array of electrode elements 102. Accordingly, the current may be increased (thereby increasing the therapeutic effect) without exceeding the safety temperature threshold at any point on the subject's skin.

In some embodiments, the anisotropic material layer 110 may be anisotropic with respect to electrical conductivity properties. In some embodiments, the anisotropic material layer 110 may be anisotropic with respect to thermal conductivity properties. In some embodiments, the anisotropic material layer 110 may be anisotropic with respect to both electrical conductivity properties and thermal conductivity properties.

The anisotropic thermal properties include directional thermal properties. Specifically, the anisotropic material layer 110 may have a first thermal conductivity in a direction that is perpendicular to its front face (skin-facing surface) 112, which may be different from a thermal conductivity of the anisotropic material layer 110 in directions that are parallel to the front face 112. For example, the thermal conductivity of the anisotropic material layer 110 in directions parallel to the front face 112 may be more than two times higher than the first thermal conductivity. In some embodiments, the thermal conductivity in the parallel directions may be more than ten times higher than the first thermal conductivity. For example, the thermal conductivity of the sheet in directions that are parallel to the front face 112 may be more than: 1.5 times, 2 times, 3 times, 5 times, 10 times, 20 times, 100 times, 200 times, or even more than 1,000 times higher than the first thermal conductivity.

The anisotropic electrical properties may include directional electrical properties.

Specifically, the anisotropic material layer 110 may have a first electrical conductivity (or, conversely, resistance) in a direction that is perpendicular to its front face 112, which may be different from an electrical conductivity (or resistance) of the anisotropic material layer 110 in directions that are parallel to the front face 112. For example, the resistance of the anisotropic material layer 110 in directions parallel to the front face 112 may be less than the first resistance. In some embodiments, the resistance in the parallel directions may be less than half of the first resistance or less than 10% of the first resistance. For example, the resistance of the anisotropic material layer 110 in directions that are parallel to the front face 112 may be less than: 75%, 50%, 40%, 30%, 20%, 10%, 5%, 1%, 0.5%, or even less than 0.1% of the first resistance.

In some embodiments (e.g., when the anisotropic material layer 110 is a sheet of pyrolytic graphite), the anisotropic material layer 110 may have both anisotropic electrical properties and anisotropic thermal properties.

The anisotropic material layer 110 may comprise graphite (e.g., a sheet of graphite or a graphite sheet). Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2010A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). In alternative embodiments, conductive anisotropic materials other than graphite may be used instead of graphite.

In some embodiments, the anisotropic material layer 110 may be a sheet of pyrolytic graphite. Thermal conductivity of pyrolytic graphite sheets in directions that are parallel to the front face 112 of those sheets may be more than 50 times higher than the thermal conductivity of those sheets in directions that are perpendicular to the front face 112. Electrical resistivity of pyrolytic graphite sheets in directions that are parallel to the front face 112 of those sheets may be less than 2% of the electrical resistivity of those sheets in directions that are perpendicular to the front face 112.

While an anisotropic material layer 110 comprised of graphite may reduce the concentration of heat and/or current at locations directly under the individual electrode elements—thus reducing the amount or severity of irritation, if any, that occurs on the subject's skin—such anisotropic material layer 110 may be relatively inflexible. In other words, when pressed against a non-flat surface, the anisotropic material layer may be at risk of cracking and/or breaking. As such, the anisotropic material layer 110 may not easily conform to a subject's body, which may be non-planar over a large area or which may bend and change shape as the subject moves. Additionally, due to a non-planar surface on a subject and/or a surface of the subject bending and changing shape, the anisotropic material layer may deform or even crack, resulting in the transducer array producing a less than desired electric field. Obtaining a flush contact surface between the electrode element and the subject's skin improves the effectiveness of the treatment. Accordingly, the anisotropic material layer 110 may include the hole 122 substantially in the center of the anisotropic material layer 110, passing through the front face 112 and the back face 114, facilitating flexibility and pliability of the anisotropic material layer 110. The hole 122 in the anisotropic material layer 110 further facilitates generation of an effective electric field and obtaining a flush contact between the transducer apparatus 100 and the subject's skin.

The transducer apparatus 100 may further include at least one layer of conductive adhesive material 116 and/or hydrogel disposed on a front facing side of the anisotropic material layer 110. In some embodiments, the at least one layer of conductive adhesive material 116 may be disposed on the front face 112 of the anisotropic material layer 110. The conductive adhesive material 116 may include the hole 122, in which case it may be aligned with the hole 122 through the anisotropic material layer 110. The at least one layer of conductive adhesive material 116 may have a biocompatible front surface. In some embodiments, there may be only a single layer of conductive adhesive material 116, and that single layer (the front layer) may be biocompatible. In some embodiments, there may be more than one layer of conductive adhesive material 116, in which case only the front layer may be biocompatible, or the front layer and one or more other layers may be biocompatible. In some embodiments, the front layer of conductive adhesive material 116 may be configured to ensure good electrical contact between the device and the body. In some embodiments, the front layer of conductive adhesive material 116 may be configured to ensure good adhesion between the anisotropic material layer 110 and the foam material layer 124. In some embodiments, the front layer of conductive adhesive material 116 may cover the entire front face 112 of the anisotropic material layer 110. The front layer of conductive adhesive material 116 may be the same size or larger than the anisotropic material layer 110. In some embodiments, the front layer of conductive adhesive material 116 comprises hydrogel. In these embodiments, the hydrogel may have a thickness between 50 and 2,000 μm. In some embodiments, the front layer of conductive adhesive material 116 may comprise a conductive adhesive composite as further disclosed herein.

The transducer apparatus 100 may further include at least one conductive material 118. The conductive material 118 may be a conductive adhesive material layer which may be positioned between the electrode elements 102 and the back face 114 of the anisotropic material layer 110. The conductive material 118 may facilitate the electrical contact between the electrode elements 102 and the back face 114 of the anisotropic material layer 110. The conductive material 118 may include the hole 122 in which case it may be aligned with the hole 122 through the anisotropic material layer 110 and the conductive adhesive material 116. In some embodiments, the conductive material 118 may be a layer of hydrogel. In other embodiments, a different conductive material (e.g., conductive grease, conductive adhesives, conductive tape, etc.) may be used. For example, the conductive material 118 may comprise a conductive adhesive composite as further disclosed herein.

In some embodiments, the at least one of the conductive adhesive material 116 and/or the conductive material 118 may be a single layer of non-hydrogel conductive adhesive such as the developmental product FLX068983-FLEXcon® OMNI-WAVE™ TT 200 BLACK H-502 150 POLY H-9 44PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Non-hydrogel conductive adhesives may comprise a waterless polymer with adhesive properties and carbon particles, powder, fibers, flakes, granules and/or nanotubes. The adhesive polymer may be, for example, an acrylic polymer or a silicone polymer, or combination thereof, which may be available as acrylic-or silicone-based carbon-filled adhesive tapes. The adhesive may additionally include one or more conductive polymers (such as, for example, polyaniline (PANI), or poly(3,4-ethylenedioxythiophene) (PEDOT), or others known in the art). The conductive filler in the at least one layer of conductive adhesive material 116 or conductive material 118 may be non-metallic. In these embodiments, the conductive adhesive may have a thickness between 10 and 2,000 μm, such as, from 20 to 1,000 μm, or 30 to 400 μm.

In some embodiments, the transducer apparatus 100 may be constructed using a pre-formed 3- (or more) layer laminate comprising the conductive material 118, the anisotropic material layer 110, and the at least one layer of conductive adhesive material 116. In some embodiments, at least one of the conductive adhesive material 116 and/or the conductive material 118 may be conductive adhesive composites as described above, and the anisotropic material layer 110 may be a thin sheet of synthetic graphite such as pyrolytic graphite, as described above. In some embodiments, the conductive adhesive material 116 and the conductive material 118 may be the same material or may be different. By way of example, in some embodiments, both the conductive adhesive material 116 and the conductive material 118 may comprise an acrylic polymer and a carbon powder filler; or both the conductive adhesive material 116 and the conductive material 118 may comprise an acrylic polymer and a carbon fiber filler. In some embodiments, the conductive adhesive material 116 may comprise an acrylic polymer and a carbon fiber filler, and the conductive material 118 may comprise an acrylic polymer and a carbon powder filler; or vice-versa. In some embodiments, one or both of the conductive adhesive material 116 and the conductive material 118 may be a hydrogel.

In some embodiments, anisotropic material layer 104 may not be included in the transducer apparatus 100. In this way, a shape of the substrate 104 may be designed to maximize the flexibility advantages described herein. The shape of the substrate 104 may also be designed to include lobes (e.g., convex contours) which contain electrode elements 102 and PCB pads 130. Additionally, the perimeter of the substrate 104 may also include concave contours that connect between the lobes, and the flexible PCB 106 may include PCB bridging connectors 135, which may have a concave shape in order to contour with the substrate 104 perimeter.

Furthermore, when viewed in a direction perpendicular to and toward the front face of the electrode elements 102, the electrode elements 102 and the PCB pads 130, when connected together, may have a shape (or shapes) which conforms to the shape of the substrate 104.

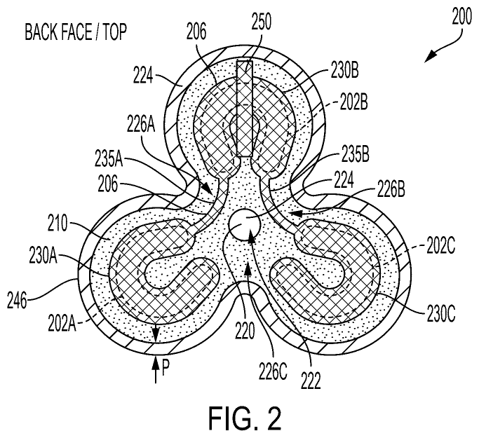

FIG. 2 depicts a top plan view (back face) of an example transducer apparatus 200 according to some embodiments. The transducer apparatus 200 in FIG. 2 may be substantially the same as the transducer apparatus 100 in FIGS. 1A to 1D, except for the shape of the electrode elements 202 and the shape of the flexible PCB 206 (including the PCB pads 230 and the PCB bridging connectors 235), and the parts in FIG. 2 use the same reference numbering as the parts in FIGS. 1A to 1D, except for the first digit changed from “1” to “2”. For ease of illustration, the substrate is not depicted in FIG. 2. The transducer apparatus 200 includes a plurality of electrode elements 202 (i.e., 202A, 202B, 202C) positioned on the anisotropic material layer 210. When viewed in a direction perpendicular to and toward the back face of the transducer apparatus 200, the electrode elements 202 may be arranged equidistant or substantially equidistant around the center 220 of the transducer apparatus 200. When viewed in a direction perpendicular to and toward the back face of the transducer apparatus 200, the electrode elements 202 may be keyhole shaped, substantially keyhole shaped, omega shaped, substantially omega shaped, arch contour loop shaped, substantially arch contour loop shaped, horseshoe shaped, or substantially horseshoe shaped. When viewed in a direction perpendicular to and toward the back face of the transducer apparatus 200, the PCB pads 230 (i.e., 230A, 230B, 230C) may have a similar shape, but larger shape, as the electrode elements 202. When viewed in a direction perpendicular to and toward the back face of the transducer apparatus 200, the PCB bridging connectors 235 (i.e., 235A, 235B) may have a similar shape as, but not as long as, the PCB bridging connectors 135. As the substrate is not depicted in FIG. 2, an offset distance P between the perimeter of the anisotropic material layer 210 and the perimeter of the perimeter portion 246 of the foam material layer 224 is illustrated in FIG. 2.

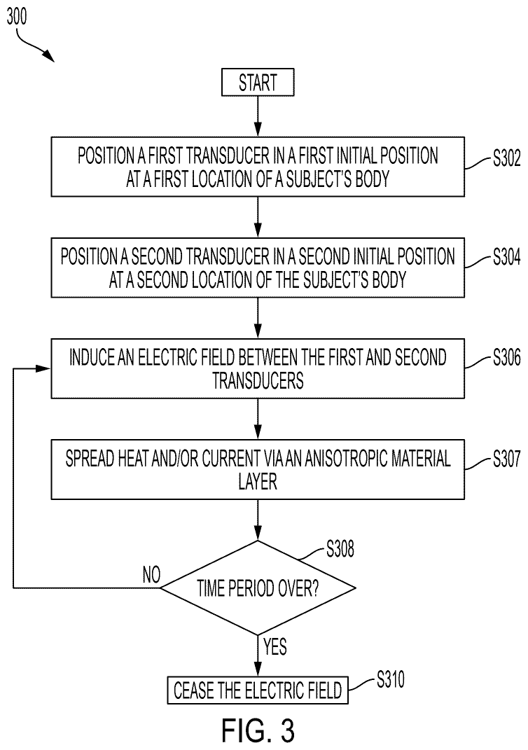

FIG. 3 is a flowchart depicting an example method 300 of applying TTFields to a subject's body according to some embodiments.

In step S302, the method 300 may include positioning a first transducer in a first initial position at a first location of the subject's body. The first transducer may be one of the example transducer apparatuses discussed herein (e.g., transducer apparatuses 100, 200). The first transducer may be affixed to the subject's body, for example, via an adhesive layer or by use of an adhesive tape or bandage. The first transducer may include an anisotropic material layer electrically coupled to the plurality of electrode elements and located between the plurality of electrode elements and the subject's body, optionally with the anisotropic material layer having a hole cutout therein, the hole being located substantially in the center of the anisotropic material layer, adjacent the electrode elements.

In step S304, the method 300 may include positioning a second transducer in a second initial position at a second location of the subject's body. The second transducer may be one of the example transducer apparatuses discussed herein (e.g., transducer apparatuses 100, 200). The second transducer may be affixed to the subject's body, for example, via an adhesive layer or by use of an adhesive tape or bandage. The second transducer may include an anisotropic material layer electrically coupled to the plurality of electrode elements and located between the plurality of electrode elements and the subject's body, optionally with the anisotropic material layer having a hole cutout therein, the hole being located substantially in the center of the anisotropic material layer, adjacent the electrode elements.

At step S306, the method 300 may include inducing an electric field between the first transducer located at the first location of the subject's body and the second transducer located at the second location of the subject's body.

At step S307, during inducing the electric field, the method 300 may include spreading heat and/or current via an anisotropic material layer from the plurality of electrode elements in a plane perpendicular to a direction from the plurality of electrode elements to the subject's body.

At step S308, the method 300 may include determining whether a first period of time has passed. Upon determining that the first period of time has passed, the method 300 proceeds to step S310. Otherwise, the method 300 returns to step S306. After inducing the electric field for more than the first period of time, the method 300 proceeds to step S310, which may include ceasing the electric field.

The invention includes other illustrative embodiments (“Embodiments”) as follows.

Embodiment 1. A transducer apparatus for delivering tumor treating fields to a subject's body, the transducer apparatus comprising: an array of electrode elements, the array configured to be positioned over the subject's body with a front face of the array facing the subject's body, the array having a back face opposite the front face; electrical connections connecting the electrode elements, the electrical connections having a front face facing the subject's body and a back face opposite the front face; and an anisotropic material layer electrically coupled to the array of electrode elements and located on a front side of the front face of the array, the anisotropic material layer comprising a front face and a back face, the back face facing the array of electrode elements, wherein, when viewed in a direction perpendicular to and toward the back face of the anisotropic material layer, the electrode elements have convex shapes with respect to a perimeter of the anisotropic material layer and the electrical connections have concave shapes with respect to the perimeter of the anisotropic material layer.

Embodiment 2. The transducer apparatus of Embodiment 1, wherein, when viewed in a direction perpendicular to and toward the back face of the anisotropic material layer, the electrical connections conform to concave shapes of the perimeter of the anisotropic material layer.

Embodiment 3. The transducer apparatus of Embodiment 1, wherein, when viewed in a direction perpendicular to and toward the back face of the anisotropic material layer, the electrode elements conform to convex shapes of the perimeter of the anisotropic material layer.

Embodiment 4. The transducer apparatus of Embodiment 1, wherein, when viewed in a direction perpendicular to and toward the back face of the anisotropic material layer, apexes of the concave shapes of the electrical connections are closer to a center of the anisotropic material layer than apexes of the convex shapes of the electrode elements.

Embodiment 5. The transducer apparatus of Embodiment 1, wherein, when viewed in a direction perpendicular to and toward the back face of the anisotropic material layer, one of the electrical connections having a concave shape electrically connects two of the electrode elements having convex shapes; or wherein, when viewed in a direction perpendicular to and toward the back face of the anisotropic material layer, three electrode elements having convex shapes are electrically connected by two electrical connections having concave shapes.

Embodiment 6. The transducer apparatus of Embodiment 1, wherein, when viewed in a direction perpendicular to and toward the back face of the anisotropic material layer, each electrode element has a same convex shape and a same size, and each electrical connection has a same concave shape and a same size.

Embodiment 7. The transducer apparatus of Embodiment 1, wherein, when viewed in a direction perpendicular to and toward the back face of the anisotropic material layer, the electrode elements are u-shaped, substantially u-shaped, c-shaped, substantially c-shaped, rounded v-shaped, substantially rounded v-shaped, jelly bean shaped, substantially jelly bean shaped, kidney bean shaped, substantially kidney bean shaped, horseshoe shaped, substantially horseshoe shaped, circular shaped, substantially circular shaped, oval shaped, substantially oval shaped, ovoid shaped, substantially ovoid shaped, keyhole shaped, substantially keyhole shaped, omega shaped, substantially omega shaped, arch contour loop shaped, or substantially arch contour loop shaped.

Embodiment 7A. The transducer apparatus of Embodiment 1, wherein, when viewed in a direction perpendicular to and toward the back face of the anisotropic material layer, three electrode elements are connected by two electrical connections.

Embodiment 7B. The transducer apparatus of Embodiment 1, wherein the electrode elements comprise a high dielectric polymer material, and the electrical connections comprise a printed circuit board.

Embodiment 8. The transducer apparatus of Embodiment 1, wherein, when viewed in a direction perpendicular to and toward the back face of the anisotropic material layer, the electrode elements and the electrical connections have shapes conforming to a shape of the anisotropic material layer.

Embodiment 9. The transducer apparatus of Embodiment 1, wherein, when viewed in a direction perpendicular to and toward the back face of the anisotropic material layer, a perimeter of the anisotropic material layer has a repeating pattern of a convex side and a concave side about a center of the anisotropic material layer.

Embodiment 10. The transducer apparatus of Embodiment 1, wherein, when viewed in a direction perpendicular to and toward the back face of the anisotropic material layer, the anisotropic material layer has a plurality of lobes about a center of the anisotropic material layer and a perimeter of each lobe of the anisotropic material layer has a convex shape with respect to the perimeter of the anisotropic material layer.

Embodiment 10A. The transducer apparatus of Embodiment 10, wherein, when viewed in a direction perpendicular to and toward the back face of the anisotropic material layer, a perimeter of the anisotropic material layer between the perimeter of each lobe of the anisotropic material layer has a concave shape with respect to the perimeter of the anisotropic material layer.

Embodiment 11. The transducer apparatus of Embodiment 10, wherein, when viewed in a direction perpendicular to and toward the back face of the anisotropic material layer, each lobe of the anisotropic material layer includes one of the electrode elements.

Embodiment 11A. The transducer apparatus of Embodiment 10, wherein, when viewed in a direction perpendicular to and toward the back face of the anisotropic material layer, one of the electrical connections is situated between two of the lobes.

Embodiment 12. The transducer apparatus of Embodiment 10 or Embodiment 11, wherein, when viewed in a direction perpendicular to and toward the back face of the anisotropic material layer, the anisotropic material layer has three lobes equidistant or substantially equidistant about the center of the anisotropic material layer.

Embodiment 12A. The transducer apparatus of Embodiment 10, wherein, when viewed in a direction perpendicular to and toward the back face of the anisotropic material layer, the lobes of the anisotropic material layer are circular shaped, substantially circular shaped, oval shaped, substantially oval shaped, ovoid shaped, or substantially ovoid shaped.

Embodiment 13. The transducer apparatus of Embodiment 1, wherein the anisotropic material layer comprises graphite.

Embodiment 13A. The transducer apparatus of Embodiment 1, wherein the anisotropic material layer comprises pyrolytic graphite, graphite foil made from compressed high purity exfoliated mineral graphite, or graphitized polymer film.

Embodiment 14. The transducer apparatus of Embodiment 1, wherein the anisotropic material layer further comprises a hole passing through the front face and the back face of the anisotropic material layer.

Embodiment 14A. The transducer apparatus of Embodiment 14, wherein, when viewed in a direction perpendicular to and toward the back face of the anisotropic material layer, the concave shapes of the electrical connections are towards the hole of the anisotropic material layer.

Embodiment 14B. The transducer apparatus of Embodiment 14, wherein, when viewed in a direction perpendicular to and toward the back face of the anisotropic material layer, the convex shapes of the electrode elements are away from the hole of the anisotropic material layer.

Embodiment 15. The transducer apparatus of Embodiment 14, wherein, when viewed in a direction perpendicular to and toward the back face of the anisotropic material layer, none of the electrode elements and none of the electrical connections cover the hole of the anisotropic material layer.

Embodiment 16. The transducer apparatus of Embodiment 1, further comprising a substrate for holding the anisotropic material layer and at least one of the electrode elements against the subject's body, wherein an outer perimeter of the substrate extends beyond an outer edge of the anisotropic material layer and, optionally, is contoured to match a shape of the outer edge of the anisotropic material layer.

Embodiment 16A. The transducer apparatus of Embodiment 16, wherein, when viewed in a direction perpendicular to and toward the back face of the anisotropic material layer, the electrode elements and the electrical connections have shapes conforming to a shape of the substrate.

Embodiment 17. A transducer apparatus for delivering tumor treating fields to a subject's body, the transducer apparatus comprising: an array of electrode elements, the array configured to be positioned over the subject's body with a front face of the array facing the subject's body, the array having a back face opposite the front face; electrical connections connecting the electrode elements, the electrical connections having a front face facing the subject's body and a back face opposite the front face; and an anisotropic material layer electrically coupled to the array of electrode elements and located on a front side of the front face of the array, the anisotropic material layer comprising a front face and a back face, the back face facing the array of electrode elements, wherein, when viewed in a direction perpendicular to and toward the back face of the anisotropic material layer, the electrode elements and the electrical connections have an undulating shape.

Embodiment 18. The transducer apparatus of Embodiment 17, wherein, when viewed in a direction perpendicular to and toward the back face of the anisotropic material layer, the electrical connections include apexes of the undulating shape closest to a center of the anisotropic material layer and the electrode elements include apexes of the undulating shape farthest from the center of the anisotropic material layer.

Embodiment 18A. The transducer apparatus of Embodiment 17, wherein, when viewed in a direction perpendicular to and toward the back face of the anisotropic material layer, the electrode elements include apexes of the undulating shape farthest from a center of the anisotropic material layer.

Embodiment 19. A transducer apparatus for delivering tumor treating fields to a subject's body, the transducer apparatus comprising: an array of electrode elements, the array configured to be positioned over the subject's body with a front face of the array facing the subject's body, the array having a back face opposite the front face; electrical connections connecting the electrode elements, the electrical connections having a front face facing the subject's body and a back face opposite the front face; and an anisotropic material layer electrically coupled to the array of electrode elements and located on a front side of the front face of the array, the anisotropic material layer comprising a front face and a back face, the back face facing the array of electrode elements, wherein, when viewed in a direction perpendicular to and toward the back face of the anisotropic material layer, the electrode elements conform to convex shapes of a perimeter of the anisotropic material layer and the electrical connections conform to concave shapes of the perimeter of the anisotropic material layer.

Embodiment 20. The transducer apparatus of Embodiment 19, wherein, when viewed in a direction perpendicular to and toward the back face of the anisotropic material layer, each electrode element has a same convex shape and a same size, and each electrical connection has a same concave shape and a same size.

Embodiment 21. A transducer apparatus for delivering tumor treating fields to a subject's body, the transducer apparatus comprising: an array of electrode elements, the array configured to be positioned over the subject's body with a front face of the array facing the subject's body, the array having a back face opposite the front face; electrical connections connecting the electrode elements, the electrical connections having a front face facing the subject's body and a back face opposite the front face; and a substrate for holding the array of electrode elements against the subject's body, the substrate comprising a plurality of lobes; wherein, when viewed in a direction perpendicular to and toward the front face of the array of electrode elements, each electrode element is positioned within a lobe of the substrate, a perimeter of the substrate comprises a concave contour which connects each of the plurality of lobes, and each of the electrical connections comprise a concave shape.

Embodiment 22. The transducer apparatus of Embodiment 21, wherein when viewed in a direction perpendicular to and toward the front face of the array of electrode elements, the electrode elements each comprise a convex shape.

Embodiment 23. The transducer apparatus of Embodiment 22, wherein, when viewed in a direction perpendicular to and toward the front face of the array of electrode elements, one of the electrical connections having a concave shape electrically connects two of the electrode elements having convex shapes; or wherein, when viewed in a direction perpendicular to and toward the front face of the array of electrode elements, three electrode elements having convex shapes are electrically connected by two electrical connections having concave shapes.

Embodiment 24. The transducer apparatus of Embodiment 21, wherein when viewed in a direction perpendicular to and toward the front face of the array of electrode elements, the electrode elements and the electrical connections have shapes conforming to a shape of the substrate.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of electrode elements, the array configured to be positioned over the subject's body with a front face of the array facing the subject's body, the array having a back face opposite the front face;
electrical connections connecting the electrode elements, the electrical connections having a front face facing the subject's body and a back face opposite the front face; and
an anisotropic material layer electrically coupled to the array of electrode elements and located on a front side of the front face of the array, the anisotropic material layer comprising a front face and a back face, the back face facing the array of electrode elements,
wherein, when viewed in a direction perpendicular to and toward the back face of the anisotropic material layer, the electrode elements have convex shapes with respect to a perimeter of the anisotropic material layer and the electrical connections have concave shapes with respect to the perimeter of the anisotropic material layer.

2. The transducer apparatus of claim 1, wherein, when viewed in a direction perpendicular to and toward the back face of the anisotropic material layer, the electrical connections conform to concave shapes of the perimeter of the anisotropic material layer.

3. The transducer apparatus of claim 1, wherein, when viewed in a direction perpendicular to and toward the back face of the anisotropic material layer, the electrode elements conform to convex shapes of the perimeter of the anisotropic material layer.

4. The transducer apparatus of claim 1, wherein, when viewed in a direction perpendicular to and toward the back face of the anisotropic material layer, apexes of the concave shapes of the electrical connections are closer to a center of the anisotropic material layer than apexes of the convex shapes of the electrode elements.

5. The transducer apparatus of claim 1, wherein, when viewed in a direction perpendicular to and toward the back face of the anisotropic material layer, one of the electrical connections having a concave shape electrically connects two of the electrode elements having convex shapes; or wherein, when viewed in a direction perpendicular to and toward the back face of the anisotropic material layer, three electrode elements having convex shapes are electrically connected by two electrical connections having concave shapes.

6. The transducer apparatus of claim 1, wherein, when viewed in a direction perpendicular to and toward the back face of the anisotropic material layer, each electrode element has a same convex shape and a same size, and each electrical connection has a same concave shape and a same size.

7. The transducer apparatus of claim 1, wherein, when viewed in a direction perpendicular to and toward the back face of the anisotropic material layer, the electrode elements are u-shaped, substantially u-shaped, c-shaped, substantially c-shaped, rounded v-shaped, substantially rounded v-shaped, jelly bean shaped, substantially jelly bean shaped, kidney bean shaped, substantially kidney bean shaped, horseshoe shaped, substantially horseshoe shaped, circular shaped, substantially circular shaped, oval shaped, substantially oval shaped, ovoid shaped, substantially ovoid shaped, keyhole shaped, substantially keyhole shaped, omega shaped, substantially omega shaped, arch contour loop shaped, or substantially arch contour loop shaped.

8. The transducer apparatus of claim 1, wherein, when viewed in a direction perpendicular to and toward the back face of the anisotropic material layer, the electrode elements and the electrical connections have shapes conforming to a shape of the anisotropic material layer.

9. The transducer apparatus of claim 1, wherein, when viewed in a direction perpendicular to and toward the back face of the anisotropic material layer, a perimeter of the anisotropic material layer has a repeating pattern of a convex side and a concave side about a center of the anisotropic material layer.

10. The transducer apparatus of claim 1, wherein, when viewed in a direction perpendicular to and toward the back face of the anisotropic material layer, the anisotropic material layer has a plurality of lobes about a center of the anisotropic material layer and a perimeter of each lobe of the anisotropic material layer has a convex shape with respect to the perimeter of the anisotropic material layer.

11. The transducer apparatus of claim 10, wherein, when viewed in a direction perpendicular to and toward the back face of the anisotropic material layer, each lobe of the anisotropic material layer includes one of the electrode elements.

12. The transducer apparatus of Embodiment 11, wherein, when viewed in a direction perpendicular to and toward the back face of the anisotropic material layer, the anisotropic material layer has three lobes equidistant or substantially equidistant about the center of the anisotropic material layer.

13. The transducer apparatus of claim 1, wherein the anisotropic material layer comprises graphite.

14. The transducer apparatus of claim 1, wherein the anisotropic material layer further comprises a hole passing through the front face and the back face of the anisotropic material layer.

15. The transducer apparatus of claim 14, wherein, when viewed in a direction perpendicular to and toward the back face of the anisotropic material layer, none of the electrode elements and none of the electrical connections cover the hole of the anisotropic material layer.

16. The transducer apparatus of claim 1, further comprising a substrate for holding the anisotropic material layer and at least one of the electrode elements against the subject's body, wherein an outer perimeter of the substrate extends beyond an outer edge of the anisotropic material layer and, optionally, is contoured to match a shape of the outer edge of the anisotropic material layer.

17. A transducer apparatus for delivering tumor treating fields to a subject's body, the transducer apparatus comprising:
an array of electrode elements, the array configured to be positioned over the subject's body with a front face of the array facing the subject's body, the array having a back face opposite the front face;
electrical connections connecting the electrode elements, the electrical connections having a front face facing the subject's body and a back face opposite the front face; and
an anisotropic material layer electrically coupled to the array of electrode elements and located on a front side of the front face of the array, the anisotropic material layer comprising a front face and a back face, the back face facing the array of electrode elements,
wherein, when viewed in a direction perpendicular to and toward the back face of the anisotropic material layer, the electrode elements and the electrical connections have an undulating shape.

18. The transducer apparatus of claim 17, wherein, when viewed in a direction perpendicular to and toward the back face of the anisotropic material layer, the electrical connections include apexes of the undulating shape closest to a center of the anisotropic material layer and the electrode elements include apexes of the undulating shape farthest from the center of the anisotropic material layer.

19. A transducer apparatus for delivering tumor treating fields to a subject's body, the transducer apparatus comprising:
an array of electrode elements, the array configured to be positioned over the subject's body with a front face of the array facing the subject's body, the array having a back face opposite the front face;
electrical connections connecting the electrode elements, the electrical connections having a front face facing the subject's body and a back face opposite the front face; and
an anisotropic material layer electrically coupled to the array of electrode elements and located on a front side of the front face of the array, the anisotropic material layer comprising a front face and a back face, the back face facing the array of electrode elements,
wherein, when viewed in a direction perpendicular to and toward the back face of the anisotropic material layer, the electrode elements conform to convex shapes of a perimeter of the anisotropic material layer and the electrical connections conform to concave shapes of the perimeter of the anisotropic material layer.

20. The transducer apparatus of claim 19, wherein, when viewed in a direction perpendicular to and toward the back face of the anisotropic material layer, each electrode element has a same convex shape and a same size, and each electrical connection has a same concave shape and a same size.
